(19) United States
(12) Patent Application Publication
(10) Pub. No.: US 2023/0165890 A1
(43) Pub. Date: Jun. 1, 2023

(54) HELP APHERESIS FOR THE TREATMENT OF SERIOUSLY ILL COVID-19 PATIENTS

(71) Applicants: Dietrich SEIDEL, Feldafing (DE); Beate R. JAEGER, Mulheim (DE)

(72) Inventors: Dietrich SEIDEL, Feldafing (DE); Beate R. JAEGER, Mulheim (DE)

(21) Appl. No.: 17/922,075

(22) PCT Filed: Apr. 30, 2021
(86) PCT No.: PCT/EP2021/061427

§ 371 Date: Oct. 28, 2022

(30) Foreign Application Priority Data
Apr. 30, 2020 (DE) 10 2020 205 557.2

(51) Int. Cl. A61K 31/727; A61K 31/737; A61P 31/14

(52) U.S. Cl. CPC A61K 31/727; A61K 31/737; A61P 31/14

(57) ABSTRACT

According to the invention, to treat severe progression of viral infections, in particular SARS-CoV-2 infections, heparin or one of its derivatives and/or another pharmaceutically acceptable polyanion is used in therapeutic apheresis, wherein a patient's blood is treated in an extracorporeal circuit such that a) blood cells are separated from plasma, b) a suitable amount of heparin/heparin derivative or pharmaceutically acceptable polyanion is added to the plasma, c) the pH of the plasma is decreased to <6 by means of a suitable buffer, d) precipitated substances are separated out, e) excess heparin and/or polyanion is adsorbed on an adsorber, f) the pH is increased back to the physiological value, and g) the treated plasma together, in parallel or successively with blood cells and, where necessary, a saline solution is reinfused into the patient.

The present invention relates to the use of heparin or its derivatives or/and other pharmaceutically acceptable polyanions in the context of a therapeutic HELP apheresis for the treatment of severe courses of viral infections, in particular covid-19 diseases.

In the era of the COVID-19 pandemic, the key question is: Which therapeutic approach should be favored to save patients who are severely ill from SARS-CoV-2 infection? What should the therapy be to avert incipient acute respiratory failure with microthrombi and inflammation of the endothelium (1,2,3) due to an exuberant immune response of the body when the first lines of immune defense have already failed? We know that SARS-CoV-2 viruses use the angiotensin-converting enzyme 2 (ACE-2) receptor and the protease TMPRSS 2 as a gateway (4) and can infect cells of the alveolar epithelium and endothelial cells of the lungs, heart, kidneys, intestine, and liver (3). Therefore, cardiac patients, hypertensives, diabetics, and patients with obesity are also particularly at risk for COVID-19, in part because receptor density is upregulated in these patients (1,2).

Pathological studies on alveolar epithelium and in endothelial cells of deceased COVID-19 patients detect the viruses in both cell types (2,3) and show a picture of a simultaneous massive inflammatory and procoagulatory activation with cell necrosis, thrombi and massive fibrinoid deposits in the microcirculation of the lung (2,3), which agglutinate the lung tissue, impede gas exchange, and thus hinder the patient's breathing. Consequently, ventilation by extracorporeal membrane oxygenation (ECMO) must also cause difficulties, just as CVVHD (continuous veno-venous hemodialysis) does not causally address this issue.

Since no drug has yet been shown to be specifically effective against the disease, there is currently no effective way to successfully treat the severely ill Covid-19 patients. Furthermore, it has now become apparent that not only the options for treating patients in an early stage of the disease, as well as those for treating patients in a highly acute, critical state are still unsatisfactory. Rather, more and more long-term impairments are becoming apparent even in patients who had only mild symptoms in the acute phase of the disease. This condition, which is now referred to as long-covoid and sometimes severely affects the lives of the affected patients, can currently only be treated relatively unspecifically and symptomatically, and patients sometimes still complain of severe symptoms months after the infection and are often unable to pursue their occupations.

The situation is similar for other severe viral infections, which also kill a tremendous number of patients worldwide each year. The inventors have therefore set themselves the task of demonstrating a different therapeutic path that takes into account the underlying mechanisms of the cellular damage observed in the patients and interrupts these mechanisms or at least slows or reduces them in such a way that the patients' demise and severe permanent health impairment can be prevented.

This task according to the invention is solved by resorting to HELP apheresis to preserve the microcirculation of the lungs and other organs, which is also already established as ultima ratio for the treatment of arteriosclerosis and in microcirculatory disorders. This method, developed by Seidel and Wieland in 1982 (5,6), was primarily aimed at patients with severe familial hypercholesterolemia (7); later, it was also successfully used in CHD (8,9), for prevention and therapy of graft vessel disease after heart transplantation (10,11,12), in peripheral arterial occlusive disease (13), acutely after strokes (14,15), and for therapy of hyperlipoproteinemia(a) (16). The procedure has anti-inflammatory and anticoagulant effects, and has proven effective in chronic as well as acute inflammatory processes of the endothelium in the micro- and macro-circulation (17).

In the method (7,17), the blood cells are first separated from the plasma in the extracorporeal circuit, a suitable amount, e.g. 400,000 units of preferably unfractionated heparin, is added to the plasma, the pH is lowered to about 4.8 to 5.25, particularly preferably 5.12, by means of a suitable buffer, particularly acetate buffer. This is close to, or in the particularly preferred form, the isoelectric point for optimal precipitation of the apolipoproteins of LDL-cholesterol, lipoprotein (a) and VLDL, which then precipitate in the precipitate filter as does fibrinogen. Downstream, the excess heparin is adsorbed and, for example, bicarbonate dialysis rebalances the pH. The treated plasma as well as the blood cells are reinfused into the patient. The duration of treatment—two hours on average—can be adapted to individual needs and extended (7,17).

This method will benefit patients with severe viral infections, especially COVID-19 patients with high probability, because:
1. HELP apheresis has no allocation problem due to the extracorporeal access and allows direct access to the entire macro- and microcirculation, removes the largest clotting protein fibrinogen from the blood (−50% in 2 h) and thus directly improves the oxygen supply in the capillaries.
2. HELP apheresis usually uses 400,000 units of unfractionated heparin in the extracorporeal circuit (7,8), which allows microthrombi to be dissolved directly without risk of heparin bleeding (15), as excess heparin is eliminated before plasma reinfusion.
3. HELP apheresis not only selectively removes 50-60% or more of fibrinogen from the blood in only 2 h of application, but also partially removes 35-50% of the precursors of both the procoagulant and fibrinolytic cascades, thus de-escalating the entire hemostaseological system (18)—with the exception of antithrombin-III, which is only 25% eliminated (19,20), further minimizing the risk for bleeding complications.
4. HELP apheresis has a direct rheological effect (13,19) by sustainably increasing myocardial (11), cerebral (14) and pulmonary blood flow rates as well as coronary flow reserve (7). This facilitates oxygen exchange in the capillaries (21).
5. HELP apheresis removes cytokines such as interleukin-6, interleukin-8 and TNF alpha and also reduces CRP concentration >50% (17). The heparin adsorber completely eliminates endotoxins and ectotoxins (19), allowing the cytokine storm and excessive inflammatory response to calm down. HELP apheresis has been successfully used in pilot studies by Bengsch et al (22) in septic multiorgan failure. The HELP procedure was successful in modified form in the EHEC epidemic in haemolytic uraemic syndrome (23).

HELP apheresis is an established commercially available system (B. Braun A G, Melsungen) that has been used clinically for 35 years. In the hands of the trained person, it is easy to handle and suitable for sustainably reducing the acute and chronic complication rates of sick patients (17).

7. HELP apheresis reduces LDL cholesterol and Lp (a) concentrations by more than 50% within 2 h (16,17), thereby improving endothelial function and possibly thereby removing circulating viruses such as coronaviruses, which use cholesterol as a vector because of their lipid coat (20).

This procedure does not remove protective IgM or IgG antibodies from the circulation and does not affect leukocyte and platelet function.

9. the long clinical experience with HELP apheresis suggests that COVID-19 patients, as well as patients suffering from other severe viral infections, will almost certainly not be harmed by therapeutic use.
10. HELP therapy has been shown in the past to be well tolerated and complementary to other therapeutic approaches, such as antiviral drugs and anticoagulants.

The embodiments of the invention are further explained in the appended claims. In particular, the present invention provides an agent which also allows to treat viral infections, especially in severe courses or/and in particular SARS-CoV-2 infections, namely in the form of a therapeutic apheresis. The agent contains heparin or one of its derivatives or/and another pharmaceutically acceptable polyanion. This agent forms the basis for the treatment within the HELP apheresis system.

In preferred embodiments, the provided agent also comprises an anion adsorber or/and a pH lowering agent. The use is then carried out by treating in the extracorporeal circulation blood of a patient in such a way that
(a) blood cells are separated from the plasma
(b) an appropriate amount of heparin/derivative or pharmaceutically acceptable polyanion is added to the plasma,
c) the pH of the plasma is lowered to <6 using a suitable buffer/pH lowering agent,
(d) substances precipitated in the process are separated,
e) excess heparin, heparin derivative or/and polyanion is adsorbed on an adsorbent,
(f) the pH is restored to the physiological value; and
(g) the treated plasma is reinfused to the patient together, in parallel or successively with blood cells and, where appropriate, a saline solution.

As explained above, the HELP process is already described in many ways in the literature, so that reference is made to such literature for further details in the treatment, for example DE 44 35 612 A1, but also literature references 5 to 21, which comprise detailed case studies or application examples. Information on treatment details available in these references can also be taken into account for the present application.

Some preferred embodiments of the use of HELP apheresis according to the invention or of the agent according to the invention are explained below. Thus, it is preferred that heparin or its derivatives are used in the form of unfractionated heparin or hydrolyzed heparin. To the extent that other pharmaceutically acceptable polyanions are used, it is again preferred to use sulfated glycosaminoglycan or a sulfated polysaccharide. A mixture of these substances with each other, or of one or both substances with heparin, may also be employed. Heparin, the heparin derivative or/and pharmaceutically acceptable polyanions, in particular as defined above, are used according to the invention in HELP apheresis and accordingly in the agent according to the invention in such an amount that from 0.001 to 10 mg/ml thereof, or 10 to 400 IU/ml in the case of heparin or its derivatives, are used based on the amount of plasma.

The pH is lowered in the HELP process to a value below 6, preferably the pH is lowered to 4.0 to 5.8, preferably 4.8 to 5.25, and particularly preferably to about 5.12 in the extracorporeal circuit. In particular, a buffer such as a citrate buffer, a lactate buffer, an acetate buffer or mixtures thereof may be present and used as the pH lowering agent in the composition according to the invention. In order to achieve a corresponding pH reduction, the pH lowering agent according to the invention is present in such an amount that in step c) of the HELP apheresis process outlined above, a dilution of the plasma with the buffer solution is carried out in a ratio of 1:5 to 5:1. In this regard, it is quite possible and may be preferred to lower the pH by adding the pH lowering agent even before adding heparin, its derivatives or/and other pharmaceutically acceptable anions. Thus, step c) of the method would be carried out before step b).

For the removal of precipitated substances, i.e. for carrying out step d) of the outlined HELP process, the precipitated substances are preferably passed over a suitable precipitate filter, in particular a filter with an average pore size of 0.01 to 1.0 μm. Alternatively, the separation can preferably be carried out by means of a flow-through centrifuge.

The anion adsorber present and used according to the invention, with the aid of which the heparin or the other acceptable polyanion with any further substances adhering thereto is separated from the blood plasma, is preferably, according to the invention, an anion exchange material which contains cations or natural, synthetic or semisynthetic polycation chains as functional groups, it being possible for polycation chains to be present in linear or branched form. Particularly preferred cations or polycations are tertiary or/and quaternary amines, in particular anion exchange materials comprising alkylaminoalkyl, dialkylaminoaryl, trialkylammoniumalkyl or trialkylammoniumaryl celluloses or/and dialkylaminoalcyl-, dialkylaminoaryl-, trialkylammoniumalkyl- or trialkylammoniumaryl-modified organic polymers or copolymers, which may optionally also be crosslinked or/and may be present in microgranular form.

The anion exchangers may preferably be based on base carrier materials of porous glass or/and silica gel coated with organic polymers or copolymers, crosslinked carbohydrates or/and organic polymers or copolymers. In particularly preferred embodiments, DEAE cellulose is used. In the use of the agent described according to the invention in the context of HELP apheresis, the original water content of the fluid is preferably restored by ultrafiltration in a further step before reinfusion of the treated plasma to the patient. Furthermore, preferably in step f) of the outlined HELP apheresis, the physiological pH is regenerated by dialysis against a suitable buffer or/and by addition of a suitable buffer, for example bicarbonate buffer.

A further object of the present invention is a method for treating viral infections, in particular severe courses of viral infections or/and SARS-COV-2 infections by means of therapeutic apheresis, which is characterized in that blood of a patient is treated in the extracorporeal circulation in such a way that
(a) blood cells are separated from the plasma
(b) an appropriate amount of heparin/derivative or pharmaceutically acceptable polyanion is added to the plasma,
c) the pH of the plasma is lowered to <6 using an appropriate buffer/pH lowering agent,
(d) substances precipitated in the process are separated,
e) excess heparin, heparin derivative or/and polyanion is adsorbed on an adsorber,
(f) the pH is restored to the physiological value; and
(g) the treated plasma is reinfused to the patient together, in parallel or successively with blood cells and, where appropriate, a saline solution.

Further preferred embodiments of this process according to the invention result from the use of the agent according to the invention described in detail above and can be carried out in the same way or in a way adapted to the treatment.

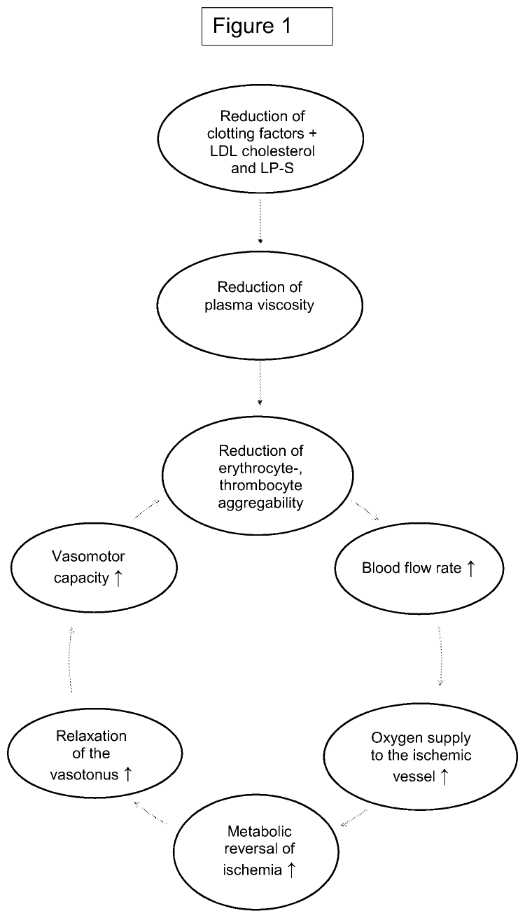

FIG. 1 shows schematically the assumed mechanism by which HELP apheresis can cause recompensation of microcirculatory disturbances. This mechanism of the HELP apheresis system should also be helpful in the treatment of SARS-CoV-2 infections.

The application of an agent according to the invention as described above, or a treatment method of the corresponding type, has proved very successful in the context so of applications to COVID-19 patients. The following embodiments once again explain the assumed mechanism of action and background, without wishing to commit the invention to corresponding mechanisms.

### Background

In COVID-19 patients with a severe, critical course as a result of infection with SARS-CoV-2, milky glass-like, interstitial clogging can be seen in the X-ray image (1), which—it is assumed—can lead to acute lung failure similar to ARDS/SIRS as a result of an excessive, no longer controllable immune response (2). Such an advanced stage of the disease only occurs when the initial antiviral lines of defence of the human body have failed: i.e. when the protective effect of interferons and secretory IgA on alveolar epithelium has not been sufficient to limit inflammation and eliminate the virus. It is currently unclear whether SARS-CoV-2 infection causes relevant viremia. This would be a prerequisite for the formation of humoral antibodies of the IgM or IgG type, which could lyse virus-infected cells in the presence of complement factors. The nature and extent of the cellular immune response to viral antigens is almost entirely T-lymphocyte dependent. Similarly, cell-mediated antibody-dependent cytotoxicity is T-cell dependent and the subject of intensive virological and cell biological research.

Ideally, it is advisable to intervene as early as possible in the inflammatory process, i.e. before—as assumed in the case of SARS-CoV-2 infection—a cytokine tsunami is triggered, which—simultaneously and inseparably—triggers an uncontrollable coagulation and inflammatory activity with the consequences already described for the microcirculation of the lung. The cytokine storm phenomenon was first described in 1973 in graft versus host disease (GVHD) after organ transplantation, later in acute respiratory distress syndrome (ARDS), sepsis, Ebola, avian influenza H5N1, smallpox, systemic inflammatory response syndrome (SIRS) and can also lead to the triggering or intensification of the disease in COVID-19.

Cytokines are peptides that act as pilots to coordinate and amplify the cellular immune response: they guide leukocytes, especially T lymphocytes and monocytes, to the site of inflammation by the viral antigen and activate them, whereupon these cells also secrete cytokines and positively feed back the immune response.https://de.wikipedia.org/wiki/Zytokinsturm-cite_note-janeway-2 In a cytokine storm, leukocytes are activated to such an extent that the immune response does not settle down. Here, high concentrations of cytokines, especially IL-1β, interleukin-6 and interleukin-8, are overexpressed. Furthermore, interleukin-1β, interleukin-6 together with TNF-alpha—the latter mainly expressed by macrophages—direct the inflammatory response and systemic effects, such as the increase of body temperature and cause capillary leakage of the microcirculation due to the increased blood flow and permeability of the capillaries. Interleukin-6 (IL-6 for short), by the nature of its complex regulation and functions in the orchestra of other cytokines and cells, plays a key role in the transition from mechanisms of innate immunity to mechanisms of acquired immunity within the inflammatory process, among others. CRP triggers II-6, IL-6 is at the same time the link to procoagulatory activation, as it is the most important trigger of fibrinogen production in the liver.

### Effects of HELP Apheresis

The anti-inflammatory effects of the HELP procedure had already been intensively investigated by Bengsch et al. (22) in the 1990s and used in pilot studies to successfully treat patients requiring intensive care with sepsis and impending multiple organ failure. In 2012, the inventors were able to free a patient with an EHEC-induced hemolytic uremic syndrome from her comatose state of consciousness within hours and from renal failure within two days (23).

In the case of Covid-19, HELP apheresis will be of immediate benefit because this extracorporeal system can simultaneously drastically reduce the trigger and the effector of the immune response: circulating cytokines, CRP and, above all, the fibrinogen concentration in the blood are reduced by 50% within two hours, and thus the rheology of the pulmonary microcirculation is immediately relieved—without reducing the erythrocyte concentration. Fibrinogen is the effector of plasmatic coagulation and is the key determinant of plasma viscosity and erythrocyte aggregability in the microcirculation ( ). In the extracorporeal system, 400,000 units of unfractionated heparin are preferably used, thus microthrombi can be dissolved directly in the extracorporeal system.

Previous studies on cardiac perfusion by positron emission tomography in heart transplanted patients have shown that the median coronary blood flow rate remains significantly elevated 24 h after a single HELP apheresis (of 2 h duration): it is still 17.5% higher at rest than before apheresis and increases by 27% under simulated stress by adenosine administration. Mainly, the lowering of fibrinogen concentration causes the rheologically significant effects in the microcirculation that facilitate oxygen exchange: plasma viscosity is reduced by an average of 19% and erythrocyte aggregability is significantly lowered by 60% (19). VEGF release and NO release are also favorably affected (17). Analogously, this improvement could also be demonstrated for the cerebral perfusion of patients with heart disease by means of Doppler examination, which can be read off from a 63% increase in CO₂ reserve capacity (19).

HELP apheresis is not limited in its application to two hours duration. The system can be recirculated for many hours—until the precipitate filter is saturated, and the filter can also be replaced on the fly—so that the fibrinogen concentration can theoretically be reduced by up to 99%, depending on requirements. Detailed preliminary studies on the influence of HELP apheresis on the kinetics of the procoagulant and fibrinolytic cascades have shown that not only fibrinogen but also the precursors of both cascades are reduced by 35-50%—with the exception of antithrombin III, which is only reduced by 25% (19,20). In sum, HELP apheresis thus causes a de-escalation of the coagulation situation of both cascades without causing bleeding because the heparin used in excess is completely adsorbed in the extracorporeal circuit of HELP apheresis (17).

The heparin adsorber, which is integrated into the apheresis, has the ability to completely eliminate endo- and ectotoxins, LP-S and LTA—of viral or bacterial origin from the patient's blood (22). Although it is currently unknown whether and to what extent toxins are also significant in the pathogenesis of SARS-CoV-2 infection, it is undisputed that the course of pulmonary infection is aggravated when toxins are present in the bloodstream. Data from the American Thoracic Society (24) also show that pneumonia is much more severe when the patient's lung microbiome is pre-colonized with, for example, Gram-negative toxin-producing bacteria.

Therefore, the use of HELP apheresis should be urgently considered at least in an advanced disease phase of a COVID-19 infection to prevent unnecessary suffering, to spare the limited intensive care capacities worldwide and to reduce costs.

In the context of the invention, HELP apheresis comprises reagents and apparatus as used in the aforementioned literatures or as described, for example, in DE 44 35 612A1. The corresponding disclosure of these literatures is therefore equally applicable to the present invention. In a particularly preferred embodiment, the HELP apheresis system of the company B.Braun Melsungen or components thereof are used and the treatment is carried out in accordance with the description of the system. Deviations within the scope of reagents, the apparatus design and treatment times can be useful and provided depending on the requirements of the individual treatment, in particular individual elements of HELP apheresis devices and methods of the prior art can be combined and interchanged with each other.

Explanations in the context of the Covid-19 disease, which is currently the focus of attention, also apply to other viral diseases in which similar mechanisms take place and which lead to similar damage. Examples of viruses causing similar mechanisms and damage are Ebola virus, RS viruses, SARS and MERS viruses, but also Coxsackie virus. Therefore, the present invention is not limited to the treatment of Covid-19, but is equally applicable to the treatment of many other severe viral infections. Thus, the present invention opens up a new treatment principle for diseases that have had little to counteract.

The following case descriptions and therapeutic examples further explain the effects of the invention:

### Therapy of Post-Acute COVID Patients With HELP Apheresis

1. The first patient is a 30-year-old nurse from a hospital in Essen. He became infected in November 2020 a. e. while on duty in the Corona ward. The first positive SARS-CoV-2 smear was detected on 2020 Nov. 24. Previously, the man was healthy and athletic. In 2017, he had pneumonia that healed. During quarantine, he developed fever, high-grade fatigue, and barely made it from bed to kitchen. He then developed a severe course of illness with protracted dyspnea that prevented him from climbing 20 steps without shortness of breath. On 2020 Dec. 20, he undertakes the first walk, but has to stop after 50-60 m. On 2021 Feb. 2, he came to us for the first time because of this complaint, as his father is a patient in our practice. We then treated him three times with HELP apheresis (2021 Feb. 2, 2021 Feb. 10 and 2021 Feb. 16). Immediately after the first apheresis, he gets better air, and can walk up stairs 40 steps relatively fast, while the respiratory rate is still accelerated. In the evening he already forgets to spray the salbutamol spray previously prescribed to him by his attending physician for support. After the 2nd apheresis he is completely symptom-free the day after, which he is pleased to report by telephone. He discontinues the medication with Foster spray which was also prescribed earlier. A few days later he can jog 20 km again. The thorax CT showed a complete healing without residuals, which continues to this day.
2. The second patient is a 24-year-old nurse who became infected while on night duty in the corona ward of a clinic in Dortmund in November 2020. Before the infection, he regularly did strength and endurance sports and played football. His arterial hypertension was treated with Ramipril. The smear had tested positive for Corona on 2020 Nov. 16. 7 h later, he develops a severe feeling of illness. His pulse rate increases to 140 per minute, he develops elevated temperatures, and loses taste and sense of smell. He sleeps on the floor because he lacks the strength to walk back to bed. For seven days, his body temperature remains at 39.2° C., then drops to 37.2° C. He takes paracetamol. The main symptoms are complete lassitude, diarrhoea and shortness of breath on the slightest physical exertion. During this period he is treated by his family doctor with Unazid 8 mg and dexamethasone for 12 days. He receives Arixtra injections. A computed tomography scan of the lungs dated 2020 Dec. 5 shows no pathological findings. He spends sleepless nights. He resumes his duties but cannot climb 2 floors without shortness of breath. Sports are out of the question. It is the shortness of breath that brought him to the surgery for the first time on 2021 Mar. 12. Two HELP aphereses 3 days apart (date: 2021 Mar. 9 and 2021 Mar. 11) had the effect that he was also symptom-free on the day after the 2nd apheresis and was able to resume his sporting activities in full until today. He informed us by telephone that he had recovered so well that we could dispense with the planned 3rd apheresis.
3. The third patient with post-acute COVID syndrome is a 53-year-old nurse who was infected in the home care service in Bochum in February 2021. Already since childhood she suffers from bronchial asthma and a heart valve does not close. The COVID infection initially causes insanely severe headaches, persistent fever of 40° C., massive fatigue and shortness of breath on the lightest exertion, and severe pain in both legs. She is treated with Revinty spray by the family doctor. A computer tomography shows a pulmonary infarction (scarring and calcifications). She also reports severe concentration problems that have persisted for over a month and have shown no improvement. After the first apheresis on 2021 Mar. 30, the patient feels a slight improvement, but it does not last. On the day after the second apheresis 2021 Apr. 6 she feels unwell. She describes an altered body perception. After the 3rd apheresis on 2021 Apr. 20 she describes a breakthrough during the apheresis: she was able to breathe deeply and freely again. In the days that followed, her physical resilience improved increasingly. She is able to go for walks again, not only in a straight line but also to master slopes. The discomfort in her legs is also much less severe.
4. The 4th patient is the husband of the 3rd patient. He will test positive for COVID-19 as part of a scheduled cataract surgery in February 2021. He is 58 years old and was diagnosed with prostate carcinoma in 2018. In the acute stage of COVID infection, he develops fever, cough, massive headache and limb pain, “like a terminal rheumatoid”. He feels tired and listless all the time. He is short of breath. He complains of difficulty concentrating, sentence breaks, forgetfulness. He can't take deep breaths. His lung function is limited. After the first apheresis on 2021 Mar. 30, he reports improved concentration and significantly fewer sentence breaks. After the 2nd apheresis on 2021 Apr. 5 he reports malaise and a “bad body feeling” one day after, but this has disappeared one day later. He reports a noticeable improvement in neurological limitations. After the 3rd apheresis on 2021 Apr. 20, both the neurological deficits and the shortness of breath have been massively alleviated.
5. The 5th patient is a 56-year-old natural scientist who had been infected in the first wave during a skiing holiday in Ischgl in March 2020 and, after the acute phase with fever, complained mainly of severe dyspnoea lasting for months with cough, poor resilience, severe concentration disorders, memory lapses, sentence interruptions, temporary paralysis and skin symptoms on the hands (blisters and peeling skin). Already during and after the first of three HELP aphereses (on 2021 Mar. 30, 2021 Apr. 6, and 2021 Apr. 15), she can breathe more freely and her concentration improves. After the 2nd apheresis she feels “like newborn”, the symptoms improve. Her skin symptoms heal. After the 3rd apheresis she is completely recovered and can ride horses and play golf again—without shortness of breath. Her ability to concentrate is also much better.
6. The 6th patient, a 30-year-old young heating engineer, complained since COVID infection in May 2020 of initially very severe headaches, loss of sense of smell persisting for 6 months as well as a persistent weakness in performance, whereby the symptoms already disappeared completely after a HELP apheresis performed on 2021 Apr. 12. A chest CT shows no pathological findings except for minor ventilation disturbances.
7. The 7th patient is a 32 year old architect. Before the COVID infection he suffers from arterial hypertension and some overweight. He is athletic. After the COVID infection in mid-Dec. 2020—presumably during a train journey—he initially develops severe headache, dizziness, earache, shortness of breath and an angina pectoris-like burning sensation in the chest, which persists for more than 14 days. He can only walk 200 m, then he has to rest. He feels “infinitely limp” and not able to bear weight. He is admitted to the Main-Kinzig-Klinikum as an inpatient on 2021 Mar. 23. The heart MRI shows a perimyocarditis with evidence of fibrosis/scarred areas subepicardially and pericardially laterally and inferolaterally. In parallel, he develops weakness of the right leg, which he drags. The MRI of the head shows two lacunar defects of the medullary camp frontal left. On 2021 Apr. 6, 2021 Apr. 12 and on 2021 Apr. 23 we perform three HELP aphereses on the patient. During the first apheresis he feels angina pectoris again, after the 1st apheresis he feels clearer in the head and less need for air. After the 2nd HELP apheresis, his symptoms improve dramatically: the headaches subside, he can put much better weight on himself and also jog again. The gait pattern is clearly better. A parallel rehabilitation measure in Bad Wildungen also helps. After the 3rd HELP apheresis on 2021 Apr. 23 he is doing much better: Email from the patient: “short update from me: I tolerated the apheresis on Friday extremely well. I feel as fit as ever. I can walk a few miles again, which is incredible! Thank you so much for that! My old life is getting closer and closer. Great. Kind regards from Bad Wildungen.”
8. The next patient, 53 years old, is the head physician of a rehabilitation clinic. Before he became infected with COVID-19 in January 2021, he was able to hike 50 km without any problems and did sports regularly. He has mixed collagenosis of mild severity. Initially after COVID infection, flu-like infection symptoms up to 39.3° C. with a wave-like course over a week. There is a non-productive cough. His resilience in everyday life is still clearly limited, climbing stairs with discomfort, also more inspiratory shortness of breath when wearing the FFP2 mask. Initial saturation fluctuations, also thoracic pain during deep inspiration. Anosmia and headache initially, improvement evident. Daytime fatigue and concentration disturbances already in the morning. New onset of restless legs and gastrointestinal irregularities. A pulmonological examination in Lippstadt on 2021 Feb. 10 shows no structural or functional damage to the lungs, but the clinical complaints are absolutely typical for COVID. An MRI examination of the head shows regular findings except for bifrontal medullary gliosis and an arachnoid cyst. The patient describes it as follows: he can no longer put weight on himself and cannot concentrate. He could not work more than 4 hours. After mowing the lawn on Easter Saturday he had been half dead for three days and had to rest for three days. He describes his symptoms as they are also described in chronic fatigue syndrome. After three aphereses on 2021 Apr. 12, 2021 Apr. 15 and 2021 Apr. 20 he reports a clear decrease of all symptoms and a striking improvement.
9. The next patient is an anaesthesia nurse in Essen. She also becomes infected while on duty in the operating theatre. The smear test from 2020 Dec. 1 is positive. She tragically infects two of her 3 children and her mother. Her mother becomes in need of intensive care as a result of the COVId-19 infection and develops ARDS syndrome and becomes permanently dialysis dependent. Her children recover. Initially, the patient develops headache, loss of smell and taste, loss of appetite, impaired thinking and concentration, and persistent shortness of breath after climbing only 1 floor of stairs. Furthermore, she reports skin rash, hair loss and black stripes in the nail (possibly embolic). We perform three aphereses on 2021 Apr. 13, 2021 Apr. 16 and 2021 Apr. 22. After the 2nd HELP apheresis, she feels clearer headed and has less shortness of breath. Two more aphereses are scheduled for next week. The patient is still on sick leave since 2021 Dec. 1.
10. The 10th patient is a 35 yr old firefighter. He became infected with COVID-19 at the end of October 2020, whether on duty or through his wife, who works as a nurse in a central emergency room, is unclear. It is also conceivable that he contracted it from his son (COVID positive but asymptomatic) who attends daycare. Initially, he has a fever of up to 40° C. for 6 days. Already after three words he gets shortness of breath. His lips become cyanotic, repeated hypertensive crises, heart rate at rest up to 160 per minute, twice presyncopal. The performance deficit persists and puts a massive strain on the patient. Before the COVID infection, he reports pedaling 270 watts on the ergometer and regularly jogging 15-20 km. Now he is unable to run 2 km. Every night he startles out of sleep one or more times, has to sit up and gasp for air. The chest CT shows left basal scarring. New Years Eve 2020, he is revaccinated against COVID-19 despite being infected while on duty, and after the 2nd vaccination, he again develops a fever that lasts for 2 days. A 1st apheresis was performed on 2020 Apr. 22. In today's phone call, his wife reports that he is breathing noticeably better and is able to sleep through the night for the first time. The 2nd apheresis is scheduled for 2021 Apr. 27.
11. The 11th patient, a former automotive mechanic, is 72 years old and contracted COVID-19 in late October 2020. His wife and son are also COVID-19 positive. The source of infection cannot be determined. When the cold symptoms do not subside after two days of bed rest, the patient calls his primary care physician, who admits him to the hospital. After one day in the corona ward, he is transferred to the intensive care unit because he has developed COVID pneumonia with acute respiratory failure. X-ray (bedside) dated 2020 Nov. 2 shows admission in decreased inspiration, further peripherally emphasized infiltrates with clinically indicated COVID infection. The patient receives systemic dexamethasone and in case of a bacterial superinfection (without evidence of pathogens) intravenous antibiotics with piperazillin and tazobactam. As the respiratory insufficiency worsens, he receives oxygen, initially intermittently as non-invasive ventilation (NIV therapy, later as nasal high flow NHF therapy). Radiographic review shows discrete loosening of known infiltration of COVID-19 pneumonia on the left compared to the prior radiograph of 2020 Nov. 2. Essentially unchanged findings with peripherally accentuated infiltration on the right. Patient remains in ICU for 17 days and recovers except for persistent hypoxemia <90% saturation, and is discharged home with negative SARS-CoV-2 PCR test and prescription for long-term oxygen therapy on 2020 Nov. 18.
One week after discharge, he suddenly develops severe lung pain and a high pulse rate of >100 min. In the emergency room he is diagnosed with pulmonary embolism and is prescribed the blood thinner Eliquis. At the end of February, the pulmonary specialist measures capillary oxygen saturation at 86 mmHg at rest, and at 71 mmHg under stress (50 watts). He is again given cortisone. Angio-CT of the lung on 2021 Feb. 11 and control CT on 2021 Mar. 25 shows unchanged chronic interstitial pneumonia without lobar pneumonic consolidations. The playout in the lung window continues to show in all lung sections a mixed pattern of mosaic-like flat lactic opacities of the lobuli with increased interstitial drawing, in each case in the lung mantle extending into the pleura. Furthermore, no consolidations, no caverns, no bronchiectasis, no pulmonary nodules, no atelectasis, no honeycomb pattern, both-sided slight pleural callosity.
On 2021 Apr. 27 we perform the first HELP apheresis: the blood in the tubes appears almost black, after 300 ml the system threatens to clot, by higher heparin doses the treatment of 3 l blood plasma can be successfully performed over 4 h at a plasma flow rate of 13 ml/min; before the apheresis the oxygen saturation in the venous blood is 25.3%—immediately after the apheresis 44.2%. The second treatment is scheduled for 2021 Apr. 30. The second treatment on 2021 Apr. 30 resulted in a further improvement of the oxygen saturation to over 52%.

### Appendix

### (a) Abbreviations:

1) non-invasive ventilation (NIV) has long been an established therapeutic option for the treatment of COPD and emphysema, which is used when there is acute or chronic exhaustion of the respiratory muscles. (Quote from Wikipedia)
2) Nasal High-flow (NHF) therapy is the application of a warmed and moistened air/oxygen mixture by means of a specialized nasal cannula. This flow of up to 60 l/min results in a reduction of the functional dead space, a washout of the airways and a small increase in respiratory pressure. This leads to an increase in respiratory efficiency with a decrease in work of breathing. Particularly in the case of pneumonia-related hypoxaemic respiratory insufficiency, an advantage over other oxygen application systems is becoming apparent. This is achieved in particular by the stable oxygenation especially at higher respiratory rates. However, the NHF is already being used successfully in other indications as well. First data show a reduction of hypercapnia. Quote from Springermedizin (J. Bräunlich and Prof. Dr. Wirtz)

### Literature:

1. Epidemiologisches Bulletin 14/2020: Schwereeinschätzung von COVID-19 mit Vergleichsdaten zu Pneumonien aus dem Krankenhaussentinel für schwere akute Atemwegserkrankungen am RKI (ICOSARI).
2. Pavan K. Bhatraju, M.D., Bijan J. Ghassemieh, M.D., Michelle Nichols, M.D., Richard Kim, M.D., Keith R. Jerome, M.D., Arun K. Nalla, Ph.D., Alexander L. Greninger, M.D., Sudhakar Pipavath, M.D., Mark M. Wurfel, M.D., Ph.D., Laura Evans, M.D., Patricia A. Kritek, M.D., T. Eoin West, M.D., M.P.H., et al. Covid-19 in Critically III Patients in the Seattle Region—Case Series NEJM 2020
3. Zsuzsanna Varga, Andreas J Flammer, Peter Steiger, Martina Haberecker, Rea Andermatt, Annelies S Zinkernagel, Mandeep R Mehra, Reto A Schueplach, Frank Ruschitzka, Holger Moch. Endothelial cell infection and endotheliitis in COVID-19.
4. W. Li, M. J. Moore, N. Vasilieva, J. Sui, S. K. Wong, M. A. Berne, M. Somasundaran, J. L. Sullivan, K. Luzuriaga, T. C. Greenough, H. Choe, M. Farzan: Angiotensin-converting enzyme 2 is a functional receptor for the SARS coronavirus. In: Nature. Volume 426, Number 6965, November 2003, pp. 450-454.
5. Seidel D, Wieland H, Ein neues Verfahren zur selektiven Messung und extrakorporalen Elimination von Low-Density Lipoproteinen, J Clin Chem Biochem (1982) 20, 684-685.
6. Seidel D, Wieland H, Ein neues Verfahren zur selektiven Messung und extrakorporalen Elimination von Low-Density Lipoproteinen, (LDL) des Plasmas, Deutsche Gesellschaft für Innere Medizin (1983) 89, 642-644.
7. Dietrich Seidel, Viktor W Armstrong, Peter Schuff-Werner, and the HELP Study Group. The HELP-LDL apheresis multicenter study, an angiographically asssessed trial into the role of LDL-apheresis in the secondary prevention of coronary heart disease, I: Evaluation of safety and cholesterol-lowering effects during the first 12 months. Eur J Clin Invest (1991), 21, 375-383.
8. Thiery J, Maximaltherapie der Hypercholesterinämie bei koronarer Herzkrankheit. Therapiewoche (1988) 38, 3424-3437.
9. Seidel D, Thiery J, Fieseler H-J, Schuff-Werner P, Eisenhauer T, Armstrong V W, Maximal Therapy of Severe Hypercholesterolemia in CHD Patients: Long term experience with the HELP LDL-apheresis in combination with HMG-CoA-reductase inhibitors. In: Gotto Jr, A. M. & Smith L. C. (Eds.) Drugs affecting lipid metabolism X. Elsevier Science Publishers B. V. (Biomedical Division) (1991) pp 299-305.
10. Park J W, Merz M, Braun P, Regression of transplant coronary artery disease during chronic low-density lipoprotein apheresis, J. of Heart and Lung Transplant, 1997, 16: 290-297.
11. Beate R. Jaeger, Bruno Meiser, Dorothea Nagel, Peter Überfuhr, Joachim Thiery, Ulrike Brandl, Werner Brückner, Wolfgang von Scheidt, Eckart Kreuzer, Gerhard Steinbeck, Bruno Reichart, Dietrich Seidel: Aggressive Lowering of Fibrinogen and Cholesterol in the Prevention of graft vessel disease after heart transplantation. Circulation 1997; 96 (suppl II): II-154-II-158.
12. a) Beate R. Jaeger: Improved survival after orthotopic heart transplantation. Atherosclerosis 2002

b) Beate R. Jaeger, Carlos A. Labarrere, Raimund Erbel: Fibrinogen Review Herz 2006
13. Walzl M, Niederkorn K, Walzl B, Horner S, Lechner H: Case reports, Reopening of Internal Carotid Artery Occlusion During Heparin-induced LDL Precipitation (H.E.L.P.) associated with improved Haemorheology: Report of 2 Cases, Wien Klin Wochenschr (1993) 105/12, 350-354.
14. Walzl M, Valetisch H, Walzl B, Lechner H: Improved cerebral blood flow in patients treated by a single heparin-induced extracorporeal LDL precipitation (H.E.L.P.), Clin Hemorheol (1994) 14/1, 27-35.
15. Walzl M, Lechner H, Walzl B, Schied G: Improved neurologic recovery of cerebral infarctions after plasmapheretic reduction of lipids and fibrinogen, Stroke (1993) 24, 1447-1451.
16. Jaeger B R, Richter Y, Nagel D, Heigl F, Vogt A, Roeseler E, Parfhofer K, Ramlow W, Koch M, Utermann G, Labarrere C A, Seidel D: Longitudinal cohort study on the effectiveness of lipid apheresis treatment to reduce high lipoprotein (a) levels and prevent major adverse coronary events, Nature Clinical Practice Vol. 6, No. 3: 229-239 (2009).
17. Dietrich Seidel, H.E.L. P. Report 1994, MMV Medizin Verlag München, ISBN 3-8208-1241-5
18. S. A. Jones, et al.: C-reactive Protein: A Physiological Activator of Interleukin-6 Receptor Shedding. In: Journal of Experimental Medicine. 189, 1999, pp. 599-604 (jem.org PDF).
19. Schuff-Werner P, Schütz E, Reitemeyer F, Oppermann M, Eisenhauser T, Armstrong V W, Kösterin H, Götze O, Seidel D: Heparin-induced Extracorporeal LDL Precipitation (H.E.L.P.), Rheological, Hemostaseological and Immunological Effects. In: Gotto A M, Richter W O, Schwandt P (eds) Treatment of Severe Hyper-cholesterolemia in the Prevention of Coronary Heart Disease. Karger, Basel (1990) 196-204.
20. Schuff-Werner P, Schütz E, Seyde W C, Eisenhauer T, Jannings G, Armstrong V W, Seidel D: Improved Haemorheology Associated With a Reduction in Plasma Fibrinogen and LDL in Patients being treated by Heparin-induced extracorporeal LDL precipitation (HELP), European Journal of Clinical Investigation, 1989, Vol 19, Issue 1, 30-37.
21. Schütz E, Schuff-Werner P, Seidel D: Einfluss der LDL-Apherese auf hämorheologische Parameter bei Patienten mit schwerer familiärer Hypercholesterinämie und KHK. In: Jung F, Kiesewetter H, Vogler E, Ehrl A M (eds) Aktuelles aus der klinischen Mikrozirkulation und Hämorheologie, so Blackwell Wissenschaft Berlin (1992) 362-370.
22. Stefan Bengsch, Boos K S, Nagel D, Seidel D, Inthorn D: Extracorporeal plasma treatment for the removal of endotoxin in patients with sepsis; clinical results of a pilot study. Shock, 2005 June; 23(6): 494-500
23. Beate R. Jaeger: Personal experience of a clinical application

1. Agent containing heparin or one of its derivatives or/and another pharmaceutically acceptable polyanion for use in the context of therapeutic apheresis in the treatment of viral infections, in particular severe courses or/and in particular SARS-CoV-2 infections.

2. Agent for use according to claim 1, characterized in that it further comprises an anion adsorber or/and a pH lowering agent.

3. Agent for use according to claim 1, characterized in that in its use in extracorporeal circulation blood of a patient is treated so that
a) blood cells are separated from plasma,
b) an appropriate amount of heparin/derivative or pharmaceutically acceptable polyanion is added to the plasma; and
c) the pH of the plasma is lowered to <6 by means of a suitable buffer,
d) precipitated substances are separated,
e) excess heparin or/and polyanion is adsorbed on an adsorber,
f) the pH is restored to the physiological value; and
g) the treated plasma is reinfused to the patient together, in parallel or successively with blood cells and, where appropriate, a saline solution.

4. Agent for use according to claim 1, characterized in that heparin or its derivatives are used in the form of unfractionated heparin or hydrolysed heparin.

5. Agent for use according to claim 1, characterized in that the other pharmaceutically acceptable anion is sulfated glucosaminoglycan or sulfated polysaccharide or a mixture of these substances with each other or with heparin.

6. Agent for use according to claim 1, characterized in that it comprises heparin or/and pharmaceutically acceptable polyanion in such an amount that from 0.001 to 10 mg/ml, or 10 to 400 IU/ml in the case of heparin or its derivatives, is used relative to the amount of plasma.

7. Agent for use according to claim 2, characterized in that it comprises the pH lowering agent in such an amount that in step c) the pH is lowered to 4.0 to 5.8, preferably to 4.8 to 5.25, and more preferably to 5.12.

8. Agent for use according to claim 2, characterized in that the pH lowering agent is a citrate buffer, a lactate buffer or an acetate buffer or a mixture thereof.

9. Agent for use according to claim 3, characterized in that it comprises the pH lowering agent in such an amount that in step c) a dilution of the plasma with the buffer solution in the ratio 1:5 to 5:1 takes place.

10. Agent for use according to claim 3, characterized in that step c) is carried out before step b).

11. Agent for use according to claim 3, characterized in that in step d) the precipitated substances are filtered off via a suitable precipitate filter, in particular a filter with an average pore size of 0.01 to 1.0 μm, or the separation is carried out by means of a flow-through centrifuge.

12. Agent for use according to claim 3, characterized in that in step e) heparin/derivatives or/and polyanions are separated by means of an anionr adsorber, wherein in particular an anion exchange material is used which contains cations or natural, synthetic or semisynthetic polycation chains as functional groups, wherein polycation chains can be present in linear or branched form.

13. Agent for use according to claim 12, characterized in that tertiary or/and quaternary amines are used as cations or polycations, in particular as anion exchange material, optionally crosslinked or/and microgranular, dialkylaminoalkyl-, dialkylaminoaryl-, trialkylammoniumalkyl- or trialkylammoniumaryl-celluloses or/and dialkylaminoalkyl-, dialkylaminoaryl-, trialkylammoniumalkyl- or trialkylammoniumaryl-modified organic polymers or copolymers are used.

14. Agent for use according to claim 2, characterized in that anion exchangers with base carrier materials of porous glass or/and silica gel coated with organic polymers or copolymers, crosslinked carbohydrates or/and organic polymers or copolymers are used.

15. Agent for use according to claim 2, which comprises DEAE cellulose as an anion exchanger or anion adsorber.

16. Agent for use according to claim 3, characterized in that in a further step, before reinfusion to the patient, the original water content of the liquid is restored by ultrafiltration.

17. Agent for use according to claim 3, characterized in that in step f) the physiological pH is regenerated by dialysis against or/and by addition of a suitable buffer, e.g. a bicarbonate buffer.

18. Method for the treatment of viral infections, in particular of severe courses of viral infections or/and of SARS-CoV-2 infections by means of therapeutic apheresis, characterized in that blood of a patient is treated in the extracorporeal circuit in such a way that
a) blood cells are separated from plasma,
b) an appropriate amount of heparin/derivative or pharmaceutically acceptable polyanion is added to the plasma; and
c) the pH of the plasma is lowered to <6 by means of a suitable buffer,
d) precipitated substances are separated,
e) excess heparin or/and polyanion is adsorbed on an adsorber,
f) the pH is restored to the physiological value; and
g) the treated plasma is reinfused to the patient together, in parallel or successively with blood cells and, where appropriate, a saline solution.

19. Method according to claim 18, characterized in that heparin or its derivatives are used in the form of unfractionated heparin or hydrolyzed heparin.

20. Method according to claim 18, characterized in that sulfated glucosaminoglycan or sulfated polysaccharide or a mixture of these substances with each other or with heparin is used as the other pharmaceutically acceptable anion.

21. Method according to claim 18, characterized in that heparin or/and pharmaceutically acceptable polyanion is used in an amount such that from 0.001 to 10 mg/ml, or 10 to 400 IU/ml in the case of heparin or its derivatives, is present relative to the amount of plasma.

22. Method according to claim 18, characterized in that the pH lowering agent is used in such an amount that in step c) the pH is lowered to 4.0 to 5.8, preferably to 4.8 to 5.25, and more preferably to 5.12.

23. Method according to claim 18, characterized in that a citrate buffer, a lactate buffer or an acetate buffer or a mixture thereof is used as the pH lowering agent.

24. Method according to claim 18, characterized in that the pH lowering agent is used in an amount such that in step c) a dilution of the plasma with the buffer solution is carried out in a ratio of 1:5 to 5:1.

25. Method according to claim 18, characterized in that step c) is carried out before step b).

26. Method according to claim 18, characterized in that in step d) the precipitated substances are filtered off via a suitable precipitate filter, in particular a filter having an average pore size of 0.01 to 1.0 μm, or the separation is carried out by means of a flow-through centrifuge.

27. Method according to claim 18, characterized in that, in step e), heparin/derivatives or/and polyanions are separated off by means of an anion adsorber, in particular an anion exchange material being used for this purpose which contains, as functional groups, cations or natural, synthetic or semisynthetic polycation chains, it being possible for polycation chains to be present in linear or branched form.

28. Method according to claim 27, characterized in that tertiary or/and quaternary amines are used as cations or polycations, in particular as anion exchange material, if desired crosslinked or/and microgranular, dialkylaminoalkyl-, dialkylaminoaryl-, trialkylammoniumalkyl- or trialkylammoniumaryl-celluloses or/and dialkylaminoalkyl-, dialkylaminoaryl-, trialkylammoniumalkyl- or trialkylammoniumaryl-modified organic polymers or copolymers are used.

29. Method according to claim 18, characterized in that anion exchangers having base carrier materials of porous glass or/and silica gel coated with organic polymers or copolymers, crosslinked carbohydrates or/and organic polymers or copolymers are used.

30. Method according to claim 18, wherein DEAE cellulose is used as the anion exchanger or anion adsorber.

31. Method according to claim 18, characterized in that in a further step, before reinfusion to the patient, the original water content of the liquid is restored by ultrafiltration.

32. Method according to claim 18, characterized in that in step f) the physiological pH is regenerated by dialysis against and/or by addition of a suitable buffer, e.g. a bicarbonate buffer.
